(19) United States
(12) Patent Application Publication
(10) Pub. No.: US 2021/0350933 A1
(43) Pub. Date: Nov. 11, 2021

(54) GENERAL AND PERSONAL PATIENT RISK PREDICTION

(71) Applicant: KONINKLIJKE PHILIPS N.V., EINDHOVEN (NL)

(72) Inventors: David Paul NOREN, Sharon, MA (US); Asif RAHMAN, Cambridge, MA (US); Bryan CONROY, Garden City South, NY (US); Minnan XU, Cambridge, MA (US); Nikhil GALAGALI, Mountain View, MA (US)

(21) Appl. No.: 17/277,619

(22) PCT Filed: Sep. 16, 2019
(86) PCT No.: PCT/EP2019/074600

§ 371 Date: Mar. 18, 2021

Related U.S. Application Data
(60) Provisional application No. 62/732,623, filed on Sep. 18, 2018.

(51) Int. Cl. G16H 50/30; G16H 50/20; G16H 10/60; G16H 50/70; G06N 20/00; G06F 17/18; A61B 5/00

(52) U.S. Cl. CPC G16H 50/30; G16H 50/20; G16H 10/60; G16H 50/70; G16H 40/67; G06F 17/18; A61B 5/7267; A61B 5/7275; G06N 20/00

(57) ABSTRACT

Various embodiments of the present disclosure are directed to a general statistical classifier (40) and a personal statistical classifier (50) for executing a patient risk prediction method. In operation, the general statistical classifier (40) may render a singular general independent vital sign risk score for a singular vital sign and/or may render plural general independent vital sign risk scores for plural vital signs. The personal statistical classifier (50) may render a singular personal vital sign risk score from an integration of a singular patient feature into the singular general independent vital sign risk score, and/or may also render plural personal independent vital sign risk scores from individual integrations of plural patient features into the singular general independent vital sign risk score, individual integrations of a singular patient feature into the plural general independent vital sign risk scores, and/or individual integrations of plural patient features into the plural general independent vital sign risk scores.

### TECHNICAL FIELD

Various embodiments described in the present disclosure relate to systems, devices, controllers and methods incorporating statistical classifiers for predicting a stable/non-deteriorating patient condition or an unstable/deteriorating patient condition.

### BACKGROUND

Over the past decade, individual care providers and health care organizations have recognized that an untreated patient unstable/deteriorating that takes place in low acuity wards is a rising problem. These patients typically go unnoticed for a variety of reasons, including low nurse to patient staffing ratios and inexperience among care providers. To address this problem, early warning score (EWS) guides have been developed that are capable of tracking subtle changes in vital signs that might otherwise go unnoticed. EWS guides (e.g., modified EWS guides and national EWS guides) have shown some efficacy in practice and have become the standard of care in some countries (e.g., the United Kingdom).

Unfortunately, adoption of EWS guides has been limited due to the high false rate alarms and overall low sensitivity. These limitations are a result in the heterogeneity among patients and current studies suggest risk assessment should be tailored to specific patient groups (e.g., respiratory illness, cardiovascular illness, septic, etc.) to be more effective.

### SUMMARY

According to the foregoing, an object of the various embodiments described in the present disclosure is to compute general independent vital sign risk scores from one or more vital signs and/or to compute a general independent vital risk scores based the general independent vital sign risk scores and one or more patient features.

For purposes of describing and claiming the present disclosure:

(1) the terms of the art of the present disclosure including, but not limited to, “vital sign”, “patient feature”, “artificial intelligence”, “statistical classifier” and “risk score” are to be broadly interpreted as known and appreciated in the art of the present disclosure and exemplary described in the present disclosure;

(2) more particularly, the term “vital sign” broadly encompass a sign as understood in the art prior to and subsequent to the present disclosure that either indicates the status of a body's vital life-sustaining functions or has been adopted in medical practice to assess the well-being of a patient. Examples of known vital signs include, but are not limited to, a heart rate, a systolic blood pressure, a respiration rate, a blood oxygen saturation (SPO2), temperature and laboratory/scientific/experimental values/measures/quantifications assessing the well-being of a patient;

(3) more particularly, the term “patient feature” broadly encompass important aspect(s) of a patient medical history and current clinical assessment. Examples of a patient feature include, but are not limited to, clinical diagnosis(ses) of disease(s) or condition(s), result(s) of laboratory test(s) and medication prescription(s);

(4) more particularly, the term “statistical classifier” broadly encompasses a machine learning model, as known in the art of the present disclosure or hereinafter conceived, that is trained in accordance with the present disclosure for predicting which category among a set of categories a new observation belongs. Examples of a statistical classifier include, but are not limited to, a Naive Bayes classifier, a logistic regression classifier, a random forest classifier and a gradient boosting classifier;

(5) more particularly, the term “risk score” broadly encompasses a score rendered by a statistical classifier that is representative of a level of risk that a new observation belongs to a category among a set of categories;

(6) the term “vital sign risk score” broadly encompasses a risk score rendered by a statistical classifier that is representative of a level of risk that a new observation of a particular vital sign belongs to a stable/non-deteriorating patient condition or an unstable/deteriorating patient condition; and

(7) the term “independent vital signal risk score” broadly encompasses a risk score for a particular vital sign rendered by a statistical classifier independent of observation(s) of other vital sign(s).

One embodiment of the present disclosure is a patient risk prediction controller employing a memory storing an artificial intelligence engine including a general statistical classifier and a personal statistical classifier. The general statistical classifier is trained on one or more vital signs to render general independent vital sign score(s), and the personal statistical classifier is trained on one or more patient features to render personal independent vital signa score(s).

The patient risk prediction controller further employs one or more processors. In operation for a singular vital sign, the processor(s) apply a trained general statistical classifier to the singular vital sign to render a singular general independent vital sign risk score. Thereafter, for a singular patient feature, the processor(s) apply a trained personal statistical classifier to the singular general independent vital sign risk score and the singular patient feature to derive a singular personal independent vital sign risk score from an integration of the singular patient feature into the singular general independent vital sign risk score. For plural patient features, the processor(s) apply a trained personal statistical classifier to the singular general independent vital sign risk score and the plural patient features to derive plural personal independent vital sign risk scores from an individual integration of each patient feature of the plural patient features into the singular general independent vital sign risk score.

Alternatively in operation for plural vital signs, the processor(s) apply a trained general statistical classifier to the plural vital signs to render plural general independent vital sign risk scores. Thereafter, for a singular patient feature, the processor(s) apply a trained personal statistical classifier to the plural general independent vital sign risk scores and the singular patient feature to derive plural personal independent vital sign risk scores from an individual integration of the singular patient feature into each general independent vital sign risk score of the plural general independent vital sign risk scores. For plural patient features, the processor(s) apply a trained personal statistical classifier to the plural general independent vital sign risk scores and the plural patient features to derive the plural personal independent vital sign risk scores from an individual integration of each patient feature of the plural patient features into each general independent vital sign risk score of the plural general independent vital sign risk scores.

A second embodiment of the present disclosure is a non-transitory machine-readable storage medium encoded with instructions for execution by one or more processors of an artificial intelligence engine including a general statistical classifier and a personal statistical classifier. Again, the general statistical classifier is trained on one or more vital signs to render general independent vital sign score(s), and the personal statistical classifier is trained on one or more patient features to render personal independent vital signa score(s).

For a singular vital sign, the encoded medium includes instructions for applying a trained general statistical classifier to the singular vital sign to render a singular general independent vital sign risk score. Thereafter, for a singular patient feature, the encoded medium further includes instructions for applying a trained personal statistical classifier to the singular general independent vital sign risk score and the singular patient feature to derive a singular personal independent vital sign risk score from an integration of the singular patient feature into the singular general independent vital sign risk score. For plural patient features, the encoded medium further includes instructions for applying a trained personal statistical classifier to the singular general independent vital sign risk score and the plural patient features to derive plural personal independent vital sign risk scores from an individual integration of each patient feature of the plural patient features into the singular general independent vital sign risk score.

Alternatively for plural vital signs, the encoded medium includes instructions for applying a trained general statistical classifier to plural vital signs to render plural general independent vital sign risk scores. Thereafter, for a singular patient feature, the encoded medium further includes instructions for applying a trained personal statistical classifier to the plural general independent vital sign risk scores and the singular patient feature to derive plural personal independent vital sign risk scores from an individual integration of the singular patient feature into each general independent vital sign risk score of the plural general independent vital sign risk scores. For plural patient features, the encoded medium further includes instructions for applying a trained personal statistical classifier to the plural general independent vital sign risk scores and the plural patient features to derive the plural personal independent vital sign risk scores from an individual integration of each patient feature of the plural patient features into each general independent vital sign risk score of the plural general independent vital sign risk scores.

A third embodiment the present disclosure is a patient risk prediction method executable by an artificial intelligence engine including a general statistical classifier and a personal statistical classifier. Again, the general statistical classifier is trained on one or more vital signs to render general independent vital sign score(s), and the personal statistical classifier is trained on one or more patient features to render personal independent vital signa score(s).

For a singular vital sign, the patient risk prediction method involves an application of a trained general statistical classifier to the singular vital sign to render a singular general independent vital sign risk score. Thereafter, for a singular patient feature, the patient risk prediction method further involves an application of a trained personal statistical classifier to the singular general independent vital sign risk score and the singular patient feature to derive a singular personal independent vital sign risk score from an integration of the singular patient feature into the singular general independent vital sign risk score. For plural patient features, the patient risk prediction method further involves an application of a trained personal statistical classifier to the singular general independent vital sign risk score and the plural patient features to derive plural personal independent vital sign risk scores from an individual integration of each patient feature of the plural patient features into the singular general independent vital sign risk score.

Alternatively for plural vital signs, the patient risk prediction method involves an application of a trained general statistical classifier to plural vital signs to render plural general independent vital sign risk scores. Thereafter, for a singular patient feature, the patient risk prediction method involves an application of a trained personal statistical classifier to the plural general independent vital sign risk scores and the singular patient feature to derive plural personal independent vital sign risk scores from an individual integration of the singular patient feature into each general independent vital sign risk score of the plural general independent vital sign risk scores. For plural patient features, the patient risk prediction method involves an application of a trained personal statistical classifier to the plural general independent vital sign risk scores and the plural patient features to derive the plural personal independent vital sign risk scores from an individual integration of each patient feature of the plural patient features into each general independent vital sign risk score of the plural general independent vital sign risk scores.

Also for purposes of describing and claiming the present disclosure:

(1) the term “controller” broadly encompasses all structural configurations, as understood in the art of the present disclosure and hereinafter conceived, of a main circuit board or an integrated circuit for controlling an application of various principles of the present disclosure as subsequently described in the present disclosure. The structural configuration of the controller may include, but is not limited to, processor(s), non-transitory machine-readable storage medium(s), an operating system, application module(s), peripheral device controller(s), slot(s) and port(s); and

(2) the terms “data” and “signals” may be embodied in all forms of a detectable physical quantity or impulse (e.g., voltage, current, magnetic field strength, impedance, color) as understood in the art of the present disclosure and as exemplary described in the present disclosure for transmitting information and/or instructions in support of applying various principles of the present disclosure as subsequently described in the present disclosure. Data/signal communication encompassed by the present disclosure may involve any communication method as known in the art of the present disclosure including, but not limited to, data/signal transmission/reception over any type of wired or wireless communication link and a reading of data uploaded to a computer-usable/computer readable storage medium.

The foregoing embodiments and other embodiments of the present disclosure as well as various features and advantages of the present disclosure will become further apparent from the following detailed description of various embodiments of the present disclosure read in conjunction with the accompanying drawings. The detailed description and drawings are merely illustrative of the present disclosure rather than limiting, the scope of the inventions of present disclosure being defined by the appended claims and equivalents thereof.

### BRIEF DESCRIPTION OF THE DRAWINGS

In order to better understand various example embodiments, reference is made to the accompanying drawings, wherein:

FIG. 1 illustrates an exemplary embodiments of an artificial intelligence engine in accordance with the principles of the present disclosure;

FIG. 2 illustrates exemplary embodiments of a patient risk prediction method in accordance with the principles of the present disclosure;

FIG. 3A illustrates an exemplary embodiment of a general statistical classifier in accordance with the principles of the present disclosure;

FIG. 3B illustrates an exemplary embodiment of a histogram in accordance with the principles of the present disclosure;

FIG. 3C illustrates an exemplary embodiment of a probability table in accordance with the principles of the present disclosure;

FIG. 4 illustrates exemplary embodiments of a general patient risk scoring method in accordance with the principles of the present disclosure;

FIG. 5 illustrates an exemplary embodiment of a personal statistical classifier in accordance with the principles of the present disclosure;

FIG. 6A illustrates exemplary embodiments of a patient feature weighting method in accordance with the principles of the present disclosure;

FIG. 6B illustrates an exemplary embodiment of personal patient risk scoring method in accordance with the principles of the present disclosure;

FIG. 7 illustrates an exemplary embodiment of a patient risk prediction controller in accordance with the principles of the present disclosure;

FIG. 8 illustrates an exemplary embodiment of a patient risk prediction system in accordance with the principles of the present disclosure; and

FIGS. 9A and 9B illustrates an exemplary embodiment of a patient risk prediction device in accordance with the principles of the present disclosure.

### DETAILED DESCRIPTION

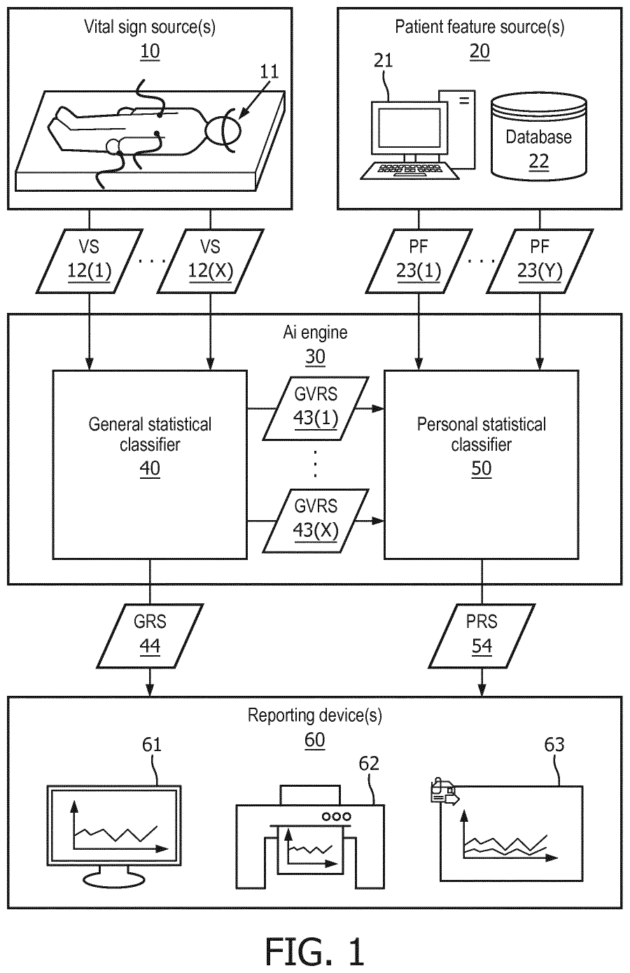

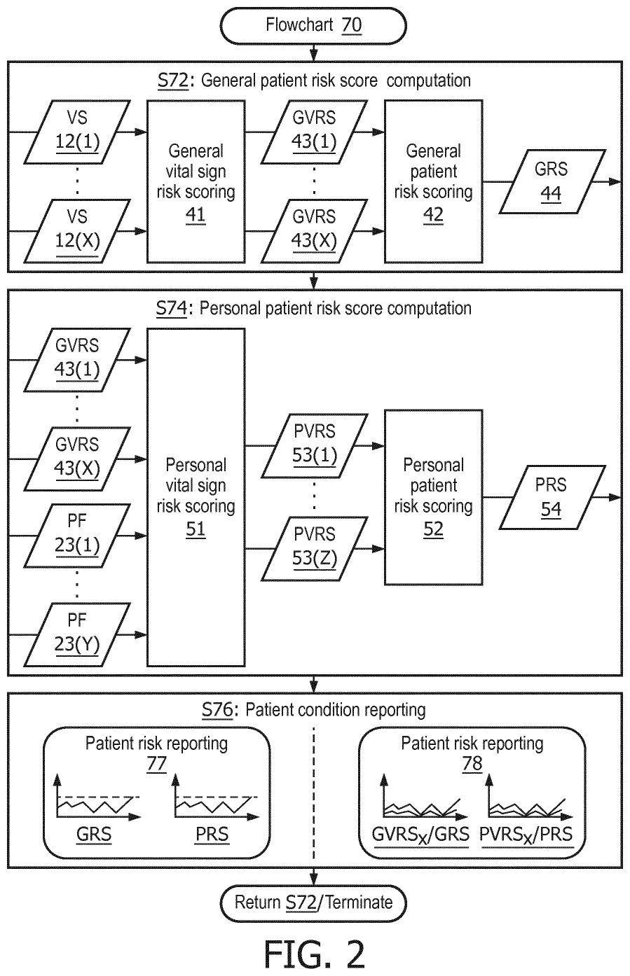

To facilitate an understanding of the present disclosure, the following description of FIGS. 1 and 2 respectively teach various embodiments of an artificial intelligence engine and a patient risk prediction method of the present disclosure. From the description of FIGS. 1 and 2, those having ordinary skill in the art of the present disclosure will appreciate how to apply the present disclosure for making and using numerous and various additional embodiments of artificial intelligence engines and patient risk prediction methods of the present disclosure.

Referring to FIG. 1, artificial intelligence engine 30 of the present disclosure employs a general statistical classifier 40 and a personal statistical classifier 50 to compute a general patient risk score (GRS) 44 from an X number of vital signs, X≥0, and/or to compute a personal patient risk score (PRS) 54 based on the vital sign(s) and a Y number of individual patient features 23, Y≥0.

For purposes of the present disclosure, vital signs 12 broadly encompass a signs that indicate the status of a body's vital life-sustaining functions. Examples of vital signs 12 include, but are not limited to, a heart rate, a systolic blood pressure, a respiration rate, a blood oxygen saturation (SPO2) and temperature.

For purposes of the present disclosure, patient features 23 broadly encompass important aspects of a patient medical history and current clinical assessment. Examples of patient features 23 include, but are not limited to, clinical diagnosis(ses) of disease(s) or condition(s), result(s) of laboratory test(s) and medication prescription(s). In practice, a singular patient feature 23 may consist of singular important aspect of the patient medical history and current clinical assessment (e.g., a singular clinical diagnosis of a disease, or a result of a singular laboratory test or a singular medication prescription), or may consist of an accumulation of plural important aspects of the patient medical history and current clinical assessment (e.g., plural clinical diagnoses of a disease or results of plural laboratory tests or plural medication prescriptions, or any combination of clinical diagnosis(ses), lab result(s) and medication prescription(s)).

Still referring to FIG. 1, in practice, general statistical classifier 40 is any type of statistical classifier as known in the art prior to and subsequent to the present disclosure that is constructed and trained in accordance with the principles of the present disclosure as exemplary described herein. Examples of various embodiments of general statistical classifier 40 include, but are not limited to, a Naive Bayes classifier, a logistic regression classifier, a random forest classifier and a gradient boosting classifier.

In a first set of embodiments, general statistical classifier 40 is constructed in accordance with the principles of the present disclosure to compute a general independent vital sign risk score (GVRS) 43 for a singular vital sign 12 (e.g., heart rate), and is trained in accordance with the principles of the present disclosure on a general patient population of the singular vital sign 12 whereby the general independent vital sign risk score 43 quantifies a probability of classifying the singular vital sign 12 as a stable/non-deteriorating patient condition (i.e., a patient condition deemed in practice harmless to a patient's health) or an unstable/deteriorating patient condition (i.e., a patient condition deemed in practice as potentially hazardous/dangerous to a patient's health). The training associated with the stable/non-deteriorating patient condition may be directed to patients recovering or recovered from a health emergency (e.g., a heart attack or a stroke) and/or a surgery (e.g., heart transplant or a coronary bypass), and the training associated with the unstable/deteriorating patient condition may be directed to deceased patients, patients transferred to a higher acuity and/or patients which required a call for a rapid response team.

For the first set of embodiments, general statistical classifier 40 may be further constructed in accordance with the principles of the present disclosure to derive the general patient risk score 44 from the singular general independent vital sign risk score 43 in any manner suitable for an informative reporting of the general patient risk score 44 quantifying a general stable/non-deteriorating patient condition or a general unstable/deteriorating patient condition. For example, the general patient risk score 44 may be equal to the singular general independent vital sign risk score 43 or a normalization of the singular general independent vital sign risk score 43.

In a second set of embodiments, general statistical classifier 40 is constructed in accordance with the principles of the present disclosure to separately compute a general independent vital sign risk score 43 for each vital sign 12 among plural vital signs 12 (e.g., heart rate, systolic blood pressure, respiration rate, blood oxygen saturation (SPO2) and temperature), and is trained in accordance with the principles of the present disclosure on a general patient population of the plural vital signs 12 whereby each general independent vital sign risk score 43 independently quantifies a probability of classifying a corresponding vital sign 12 as a stable/non-deteriorating patient condition (i.e., a patient condition deemed in practice as harmless to a patient's health) or an unstable/deteriorating patient condition (i.e., a patient condition deemed in practice as potentially hazardous/dangerous to a patient's health). Again, the training associated with the stable/non-deteriorating patient condition may be directed to patients recovering or recovered from a health emergency (e.g., a heart attack or a stroke) and/or a surgery (e.g., heart transplant or a coronary bypass), and the training associated with the unstable/deteriorating patient condition may be directed to deceased patients, patients transferred to a higher acuity and/or patients which required a call for a rapid response team.

For the second set of embodiments, general statistical classifier 40 may be further constructed in accordance with the principles of the present disclosure to derive the general patient risk score 44 from the plural general independent vital sign risk scores 43 in any manner suitable for an informative reporting of the general patient risk score 44 quantifying a general stable/non-deteriorating patient condition or a general unstable/deteriorating patient condition. For example, the general patient risk score 44 may be an aggregation of the plural general independent vital sign risk scores 43 in the form of a summation of the plural general independent vital sign risk scores 43, or a normalization of a summation of the plural general independent vital sign risk scores 43.

Still referring to FIG. 1, in practice, personal statistical classifier 50 is any type of statistical classifier as known in the art prior to and subsequent to the present disclosure that is constructed in accordance with the principles of the present disclosure as exemplary described herein. Examples of various embodiments of personal statistical classifier 50 include, but are not limited, a linear regression classifier, a logistic regression based classifier, a polynomial regression based classifier, a stepwise regression based classifier, a ridge regression based classifier, a lasso regression based classifier and a ElasticNet regression based classifier.

In a first set of embodiments, personal statistical classifier 40 is constructed in accordance with the principles of the present disclosure to derive a singular personal independent vital sign risk score (not shown in FIG. 1, PVRS 53 shown in FIG. 2) from an integration of a singular patient feature 23 (e.g., a clinical diagnosis, a laboratory test result or a medication prescription) into a singular general independent vital sign risk score 43 (e.g., a general heart rate risk score), and is trained to the singular patient feature 23 using a form of regression. For example, the personal independent vital sign risk score may be an integration of the singular patient feature 23 into the singular general independent vital sign risk score 43 in the form of a product of a weighted function of the singular patient feature 23 and the singular general independent vital sign risk score 43, or a normalization of a product of a weighted function of the singular patient feature 23 and the singular general independent vital sign risk score 43.

For purposes of the present disclosure, a weighted function of the singular patient feature 23 broadly encompasses a quantification of the singular patient feature 23 that further personally refines the singular general independent vital sign risk score 43 as a probability of classifying the singular vital sign 12 as a stable/non-deteriorating patient condition or an unstable/deteriorating patient condition. In practice, the weighted function may be simple (e.g., a binary number indicating an absence or a presence of a particular clinical diagnosis, a particular lab result or a particular medication prescription) or complex (e.g., a multivariate expression of various categories of a clinical diagnosis, numerous test ranges of lab results and various dosages of a medication prescription). For example, the weighted function of the singular patient feature 23 may be a product of simple or complex coefficient(s) and the singular patient feature 23.

For the first set of embodiments, the personal statistical classifier 50 is further constructed in accordance with the principles of the present disclosure to derive the personal patient risk score 54 from the singular personal independent vital sign risk score in any manner suitable for an informative reporting of the personal patient risk score 54 quantifying a personal stable/non-deteriorating patient condition or a personal unstable/deteriorating patient condition. For example, the personal patient risk score 54 may be equal to the singular personal independent vital sign risk score or a normalization of the singular personal independent vital sign risk score.

In a second set of embodiments, personal statistical classifier 40 is constructed in accordance with the principles of the present disclosure to derive plural personal independent vital sign risk scores (not shown in FIG. 1, PVRS 53 shown in FIG. 2) from an independent integration of plural patient features 23 (e.g., a clinical diagnosis, a laboratory test and a medication prescription) into a singular general independent vital sign risk score 43 (e.g., a general heart rate risk score) and is trained to the plural patient features 23 using a form of regression. For example, the singular personal independent vital sign risk scores may be an independent integration of each of the plural patient features 23 into the singular general independent vital sign risk score 43 in the form of a separate product of a weighted function of each of the plural patient features 23 and the singular general independent vital sign risk score 43, or a normalization of a separate product of a weighted function of each of the plural patient features 23 and the singular general independent vital sign risk score 43.

For purposes of the present disclosure, a weighted function of each patient feature 23 broadly encompasses a quantification of each patient feature 23 that further personally refines the singular general independent vital sign risk score 43 as a probability of classifying the singular vital sign 12 as a stable/non-deteriorating patient condition or an unstable/deteriorating patient condition. Again, in practice, the weighted function may be simple (e.g., a binary number indicating an absence or a presence of a particular clinical diagnosis, a particular lab result or a particular medication prescription) or complex (e.g., a multivariate expression of various categories of a clinical diagnosis, numerous test ranges of lab results and various dosages of a medication prescription). For example, the weighted function of each of the plural patient features 23 may be a product of simple or complex coefficient(s) and a corresponding patient feature 23.

For the second set of embodiments, the personal statistical classifier 50 is further constructed in accordance with the principles of the present disclosure to derive the personal patient risk score 54 from the plural personal independent vital sign risk scores in any manner suitable for an informative reporting of the personal patient risk score 54 quantifying a personal stable/non-deteriorating patient condition or a personal unstable/deteriorating patient condition. For example, the personal patient risk score 54 may be an aggregation of the plural personal independent vital sign risk scores in the form of a summation/a product of the plural personal independent vital sign risk scores or a normalization of a summation/a product of the plural personal independent vital sign risk scores.

In a third set of embodiments, personal statistical classifier 40 is constructed in accordance with the principles of the present disclosure to derive plural personal independent vital sign risk scores (not shown in FIG. 1, PVRS 53 shown in FIG. 2) from an independent integration of a singular patient feature 23 (e.g., a diagnosis, a laboratory test or a medication) into plural general independent vital sign risk scores 43 (e.g., a general heart rate risk score and a general blood pressure risk score). For example, each of the plural personal independent vital sign risk score may be an integration of the singular patient feature 23 into one of the plural general independent vital sign risk scores 43 in the form of a separate product of a weighted function of the singular patient feature 23 and each general independent vital sign risk score 43, or a normalization of separate product of a weighted function of the singular patient feature 23 and each general independent vital sign risk score 43.

For purposes of the present disclosure, a weighted function of the singular patient feature 23 broadly encompasses a quantification of the singular patient feature 23 that further personally refines each general independent vital sign risk score 43 as a probability of classifying each vital sign 12 as a stable/non-deteriorating patient condition or an unstable/deteriorating patient condition. Again, in practice, the weighted function may be simple (e.g., a binary number indicating an absence or a presence of a particular diagnosis, a particular lab result or a particular medication) or complex (e.g., a multivariate expression of various categories of a diagnosis, numerous test ranges of lab results and a number of a particular type of medication). For example, the weighted function of the singular patient feature 23 may be a product of simple or complex coefficient(s) and the singular patient feature 23.

For the third set of embodiments, the personal statistical classifier 50 is further constructed in accordance with the principles of the present disclosure to derive the personal patient risk score 54 from the plural personal independent vital sign risk score in any manner suitable for an informative reporting of the personal patient risk score 54 quantifying a personal stable/non-deteriorating patient condition or a personal unstable/deteriorating patient condition. For example, the personal patient risk score 54 may be an aggregation of the plural personal independent vital sign risk scores in the form of a summation/a product of the plural personal independent vital sign risk scores or a normalization of a summation/a product of the plural personal independent vital sign risk scores.

In a fourth set of embodiments, personal statistical classifier 40 is constructed in accordance with the principles of the present disclosure to derive plural personal independent vital sign risk scores (not shown in FIG. 1, PVRS 53 shown in FIG. 2) from an independent integration of plural patient features 23 (e.g., a diagnosis, a laboratory test and a medication) into plural general independent vital sign risk scores 43 (e.g., a general heart rate risk score and a general blood pressure risk score). For example, the personal independent vital sign risk scores may be an independent integration of each patient feature 23 into each general independent vital sign risk score 43 in the form of a separate product of a weighted function of each patient feature 23 and each general independent vital sign risk score 43, or a separate logarithmic product of a weighted function of each patient feature 23 and each general independent vital sign risk score 43,

For purposes of the present disclosure, a weighted function of each patient feature 23 broadly encompasses a quantification of each patient feature 23 that further personally refines each general independent vital sign risk score 43 as a probability of classifying each vital sign 12 as a stable/non-deteriorating patient condition or an unstable/deteriorating patient condition. Again, in practice, the weighted function may be simple (e.g., a binary number indicating an absence or a presence of a particular clinical diagnosis, a particular lab result or a particular medication prescription) or complex (e.g., a multivariate expression of various categories of a clinical diagnosis, numerous test ranges of lab results and various dosages of a medication prescription). For example, the weighted function of each of the plural patient features 23 may be a product of simple or complex coefficient(s) and a corresponding patient feature 23.

For the fourth set of embodiments, the personal statistical classifier 50 is further constructed in accordance with the principles of the present disclosure to derive the personal patient risk score 54 from the personal independent vital sign risk scores in any manner suitable for an informative reporting of the personal patient risk score 54 quantifying a personal stable/non-deteriorating patient condition or a personal unstable/deteriorating patient condition. For example, the personal patient risk score 54 may be an aggregation of the plural personal independent vital sign risk scores in the form of a summation/product of the plural personal independent vital sign risk scores or a normalization of a summation/a product of the plural personal independent vital sign risk scores.

Referring to FIGS. 1 and 2, in operation, artificial intelligence engine 30 executes a flowchart 70 representative of a patient risk prediction method of the present disclosure.

During a stage S72 of flowchart 70, artificial intelligence engine 30 receives a singular vital sign 12 from a vital sign source 10, or plural vital signs 12 from one or more vital sign sources 10. In practice, vital sign sources 10 may be any type of source capable of sensing, detecting or otherwise monitoring a vital sign of a patient 11. Examples of vital sign sources 10 include, but are not limited to, heart rate sensors, electrocardiograms, blood pressure sensors, respiratory rate sensors, pulse oximeters and thermometers. In practice, the vital sign(s) 12 may be communicated by techniques known in the art prior to and subsequent the present disclosure at any time suitable for ascertaining a condition of patient 11 (e.g., in real-time or post-study).

Upon receipt of a singular vital sign 12, general statistical classifier 40 executes a general vital sign risk scoring 41 of the singular vital sign 12 to render a singular general independent vital sign risk score 43 as previously described in the present disclosure. Subsequently, general statistical classifier 40 executes a general patient risk scoring 42 to compute general patient risk score 44 as previously described in the present disclosure. For example, during scoring 41, general statistical classifier 40 may implement a Naive Bayes classification, a logistic regression classification, a random forest classification or a gradient boosting classification of the singular vital sign 12 to render the singular general independent vital sign risk score 43. Subsequently, during scoring 42, general statistical classifier 40 may compute general patient risk score 44 as the singular general independent vital sign risk score 43.

Upon receipt of plural vital signs 12, general statistical classifier 40 executes general vital sign risk scoring 41 of the plural vital signs 12 to render plural general independent vital sign risk scores 43 as previously described in the present disclosure. Subsequently, general statistical classifier 40 executes a general patient risk scoring 42 to compute general patient risk score 44 as previously described in the present disclosure. For example, during scoring 41, general statistical classifier 40 may individually implement a Naive Bayes classification, a logistic regression classification, a random forest classification or a gradient boosting classification of each of the plural vital signs 12 to render the plural general independent vital sign risk scores 43. Subsequently, during scoring 42, general statistical classifier 40 may implement a summation/a product of the plural general independent vital sign risk scores 43 to compute general patient risk score 44.

Still referring to FIGS. 1 and 2, during a stage S74 of flowchart 70, artificial intelligence engine 30 receives a singular patient feature 23 from a patient feature source 20, or plural patient features 23 from one or more patient feature sources 20. In practice, patient feature sources 20 may be any type of source capable of a downloading/uploading or otherwise transferring patient feature(s) 12 to artificial intelligence engine 30. Examples of patient features source(s) 20 include, but are not limited to, a workstation 21 located at a health care facility or a health care provider office, and a database 22 installed within a remote medical data reporting site. In practice, the patient feature(s) 23 may be communicated by techniques known in the art prior to and subsequent to the present disclosure at any time suitable for ascertaining a condition of patient 11 (e.g., in real-time or post-study).

Upon receipt of a singular patient feature 23, personal statistical classifier 50 executes a personal vital sign risk scoring 51 of the singular patient feature 23 and a general independent vital sign risk score 43 or plural general independent vital sign risk scores 43, whichever is applicable, to render a singular personal independent vital sign risk score 53 or plural personal independent vital sign risk scores 53, respectively, as previously described in the present disclosure. Subsequently personal statistical classifier 50 executes a personal patient risk scoring 52 to compute personal patient risk score 54 as previously described in the present disclosure. For example, during scoring 51, personal statistical classifier 50 may implement a weighted function of singular patient feature 23 and a compute a product of the weighted function of the single patient feature 23 and the singular general independent vital sign risk score 43 or of the plural general independent vital sign risk score(s) 43, whichever is applicable, to render the singular personal independent vital sign risk score 53 or the plural personal independent vital sign risk scores 53. Subsequently, during scoring 52, personal statistical classifier 50 may equate the singular personal independent vital sign risk score 53 as personal patient risk score 54 or may implement a summation of the plural personal independent vital sign risk scores 53, whichever is applicable.

Upon receipt of plural patient features 23, personal statistical classifier 50 executes a personal vital sign risk scoring 51 of the plural patient features 23 and a general independent vital sign risk score 43 or plural general independent vital sign risk scores 43, whichever is applicable, to render the plural personal independent vital sign risk scores 53 as previously described in the present disclosure. Subsequently personal statistical classifier 50 executes a personal patient risk scoring 52 to compute the personal patient risk score 54 as previously described in the present disclosure. For example, during scoring 51, personal statistical classifier 50 may generate a weighted function of each of the plural patient features 23 and a compute an individual product of the weighted function of each of the plural patient feature 23 and the singular general independent vital sign risk score 43 or of the plural general independent vital sign risk score(s) 43, whichever is applicable, to render the plural personal independent vital sign risk scores 53. Subsequently, during scoring 52, personal statistical classifier 50 may implement a summation of the plural personal independent vital sign risk scores 53 to compute the personal patient risk score 54.

Still referring to FIGS. 1 and 2, during a stage S76 of flowchart 70, artificial intelligence engine 30 communicates general patient risk score 44, if applicable, and personal patient risk score 54 to reporting devices 60 as known in the art of the present disclosure. Examples of reporting devices 60 include, but are not limited to, a monitor 61 for a visual reporting, a plotter 62 for a graphical reporting or an email 63 for a textual reporting. In one embodiment, artificial intelligence engine 30 executes a patient risk reporting 77 as shown in FIG. 2 involving a charting of general patient risk score 44 relative to an unstable/deteriorating threshold (dashed line) and/or a charting of personal patient risk score 44 relative to an unstable/deteriorating threshold (dashed line).

In a second embodiment, artificial intelligence engine 30 executes a patient risk reporting 78 as shown in FIG. 2 involving a charting of general patient risk score 44 relative to one or more of the general independent vital sign risk scores and/or a charting of personal patient risk score 44 relative to one or more of the personal independent vital sign risk scores.

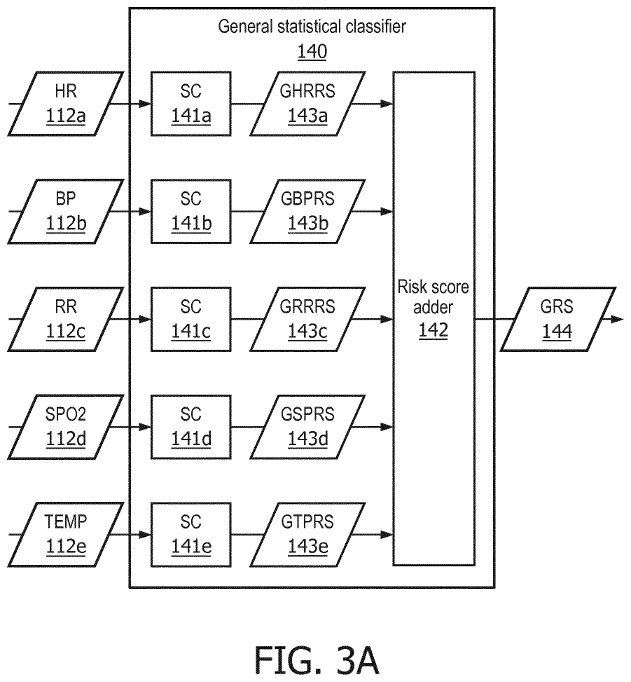

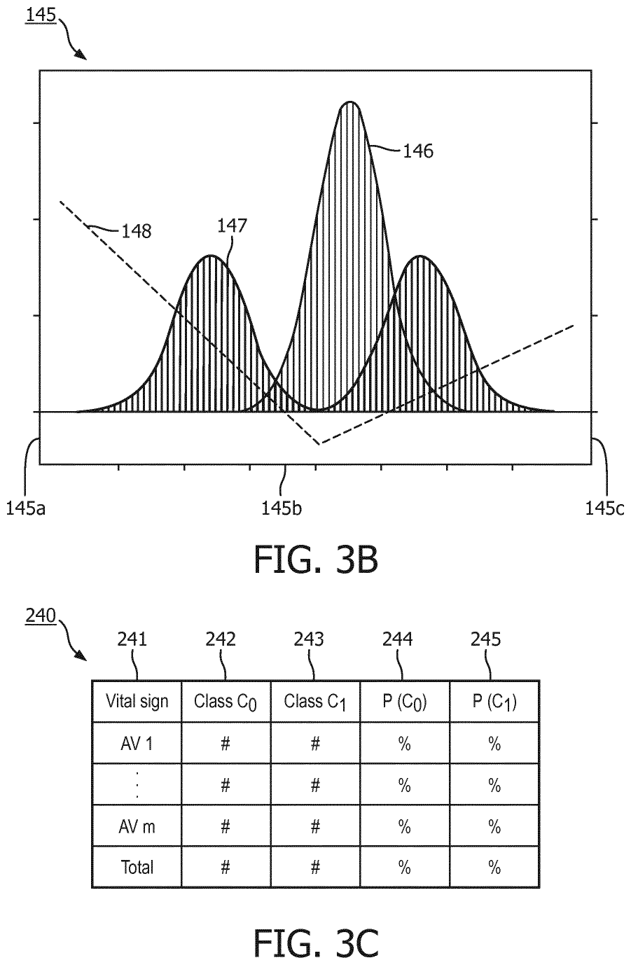

To facilitate a further understanding of the present disclosure, the following description of FIGS. 3A-6B respectively teach various embodiments of the general statistical classifier of FIG. 1 and the personal statistical classifier of FIG. 1. From the description of FIGS. 3A-6B, those having ordinary skill in the art of the present disclosure will appreciate how to apply the present disclosure for making and using numerous and various additional embodiments of general statistical classifiers and personal statistical classifiers of the present disclosure.

For clarity purposes, the following description of FIGS. 3A-6B is in the context of vital signs including a heart rate, a systolic blood pressure, a respiratory rate, a systolic blood pressure, a respiration rate, a blood oxygen saturation (SPO2) and temperature, and further in the context of patient features including a singular cardiac clinical diagnosis, results of a singular cardiac laboratory test and a singular prescribed cardiac medication. Nonetheless, those having ordinary skill in the art of the present disclosure will appreciate how to apply description the present disclosure for making and using numerous and various additional embodiments of general statistical classifiers and personal statistical classifiers of the present disclosure in the context of a different listing of vital signs (e.g., more or less vital signs) and a further in the context of a different listing of patient features (e.g., additional or different patient features).

Referring to FIG. 3A, one embodiment of general statistical classifier 40 (FIG. 1) is general statistical classifier 140 employing a parallel network of five (5) statistical classifier (SC) 141a-141e and a risk score adder 142.

In operation, statistical classifier 141a is constructed and trained to input heart rate (HR) signal 112a to thereby render a general heart rate risk score (GHRRS) 143a.

Statistical classifier 141b is constructed and trained to input a blood pressure (BP) signal 112b to thereby render a general blood pressure risk score (GBPRS) 143b.

Statistical classifier 141c is constructed and trained to input a respiratory rate (RR) signal 112c to thereby render a general respiratory rate risk score (GRRRS) 143c.

Statistical classifier 141d is constructed and trained to input a blood oxygen saturation (SPO2) signal 112d to thereby render a general blood oxygen saturation risk score (GSPRS) 143d.

Statistical classifier 141e is constructed and trained to input a temperature (TEMP) signal 112e to thereby render a general temperature risk score (GTPRS) 143e.

In practice, statistical classifiers 141a-141e may implement a statistical classifier as known in the art prior to and subsequent to the present disclosure that is constructed and trained in accordance with the principles of the present disclosure to render the plural general independent vital sign risk scores 143a-143e respectively for plural vitals sign 112a-112e. Examples of various embodiments of statistical classifiers 141a-141e include, but are not limited to, a parallel network of Naive Bayes classifier, a parallel network of logistic regression classifiers, a parallel network of random forest classifier and a parallel network of gradient boosting classifiers.

For clarity purposes, the following description of the parallel network of statistical classifiers 141a-141e will be as a parallel network of Naive Bayes classifiers. Nonetheless, from the description of the parallel network of Naive Bayes classifiers, those having ordinary skill in the art of the present disclosure will appreciate how to apply description the present disclosure for making and using numerous and various additional embodiments of the parallel network of statistical classifiers 141a-141e including, but not limited to, a parallel network of logistic regression classifiers trained in accordance with the present disclosure via logistic/sigmoid function(s) as known in the art prior to and subsequent to the present disclosure, a parallel network of random forest classifiers trained in accordance with the present disclosure via decision trees as known in the art prior to and subsequent to the present disclosure and a parallel network of gradient boosting classifiers trained in accordance with the present disclosure on prediction models as known in the art prior to and subsequent to the present disclosure.

In one embodiment of general statistical classifier 140 as shown in FIG. 3B, each statistical classifier 141a-141e generates a training histogram 145 of continuous values having a density axis 145a, a vital sign axis 145b and a risk curve axis 145c. A mixture model (e.g., a gaussian model, a log-normal mixture, an exponential mixture, an alpha mixture and a beta mixture) is used to fit a stable/non-deteriorating class C₀, distribution 146 of stable/non-deteriorating training values of an assigned vital sign and an unstable/deteriorating class C₁ distribution 147 of unstable/deteriorating training values of the assigned vital sign plotted within the training histogram 145. A vital sign risk curve 148 is calculated in accordance with the following log odds ratio equation [1], the following normalized probability equation [2] of the following normalized probability equation [3]:

| | |
|---|---|
| log (P(Xᵢ \| C₁)/P(Xᵢ \| C₀)) | [1] |
| log (P(Xᵢ \| C₀)/P(Xᵢ)) | [2] |
| log (P(Xᵢ \| C₁)/P(Xᵢ)) | [3] |

For equations [1]-[3], P(Xᵢ|C₀) is the probability of observing the assigned vital sign for the stable/non-deteriorating class C₀, P(Xᵢ|C₁) is the probability of observing the assigned vital sign for the unstable/deteriorating class C₁, and P(Xᵢ) is the probability of observing the assigned vital sign.

In a second embodiment of general statistical classifier 140 as shown in FIG. 3C, each statistical classifier 141 generates a training probability table 240 of discrete values including a column 241 of attribute values of a vital sign (e.g., attribute values for a heart rate as established by an early warning score guide), a column of 242 number of occurrences of a stable/non-deteriorating condition C₀ of each attribute value of the vital sign, a column of 243 number of occurrences of an unstable/deteriorating condition C₁ of each attribute value of the vital sign, a column 244 of a probability a stable/non-deteriorating condition C₀ of attribute value of the vital sign in accordance with one of the aforementioned equations [1]-[3], and a column 245 of a probability an unstable/deteriorating condition C₁ of attribute value of the vital sign in accordance with one of the aforementioned equations [1]-[3].

In practice, risk score adder 142 is any type of adder as known in the art prior to and subsequent to the present disclosure that is constructed accordance with the principles of the present disclosure to compute a general patient risk score 144 as a summation of the plural general independent vital sign risk scores 143a-143e.

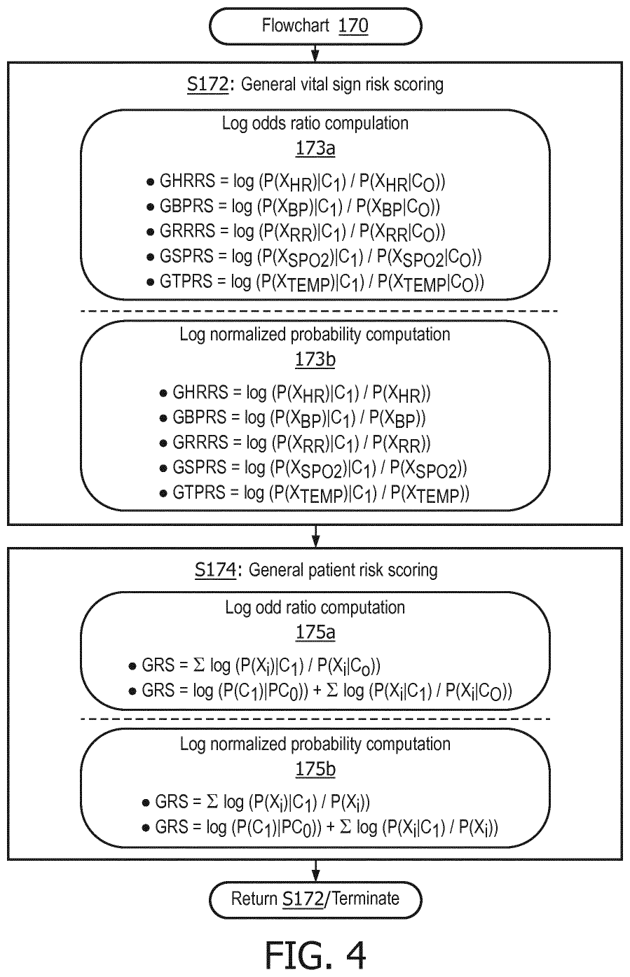

Referring to FIGS. 3A and 4, in operation, general statistical classifier 140 implements a flowchart 170 representative of a general patient risk scoring computation stage S72 of FIG. 2.

During a stage S172 of flowchart 170, statistical classifiers 141a-141e independently renders general independent vital sign risk scores 143a-143e respectively for vital sign 112a-112e.

For a log odds ratio embodiment 173a, statistical classifiers 141a-141e independently renders the plural general independent vital sign risk scores 143a-143e respectively for vital sign 112a-112e in accordance with the following equations [4]-[8]:

| | |
|---|---|
| GHHRS = log (P(X_{HR} \| C₁)/P(X_{HR} \| C₀)) | [4] |
| GBPRS = log (P(X_{BP} \| C₁)/P(X_{BP} \| C₀)) | [5] |
| GRRRS = log (P(X_{RR} \| C₁)/P(X_{RR} \| C₀)) | [6] |
| GSPRS = log (P(X_{SPO2} \| C₁)/P(X_{SPO2} \| C₀)) | [7] |
| GTPRS = log (P(X_{TEMP} \| C₁)/P(X_{TEMP} \| C₀)) | [8] |

For a normalized probability embodiment 173b, statistical classifiers 141a-141e independently renders general independent vital sign risk scores 143a-143e respectively for vital sign 112a-112e in accordance with the following equations [9]-[13] for either the stable/non-deteriorating class C₀ or the unstable/deteriorating class C₁:

| | |
|---|---|
| GHHRS = log (P(X_{HR} \| C₁)/P(X_{HR})) | [9] |
| GBPRS = log (P(X_{BP} \| C₁)/P(X_{BP})) | [10] |
| GRRRS = log (P(X_{RR} \| C₁)/P(X_{RR})) | [11] |
| GSPRS = log (P(X_{SPO2} \| C₁)/P(X_{SPO2})) | [12] |
| GTPRS = log (P(X_{TEMP} \| C₁)/P(X_{TEMP})) | [13] |

During a stage S174 of flowchart 170, risk score adder 142 compute general patient risk score 144 as a summation of general independent vital sign risk scores 143a-143e.

For a log odds ratio embodiment 175a, risk score adder 142 computes general patient risk score 144 as summation of the plural general independent vital sign risk scores 143a-143e in accordance with the following equation [14a] or the following equation [14b]:

| | |
|---|---|
| GRS = Σlog (P(Xᵢ \| C₁)/P(Xᵢ \| C₀)) | [14a] |
| GRS = log (P(C₁)/P(C_{O})) + Σlog (P(Xᵢ \| C₁)/P(Xᵢ \| C₀)) | [14b] |

For equation 14[b], log (P(C₁)/P(C₀)) represents a term for biasing the GRS by the overall prevalence of unstable/deteriorating class C₁.

For a normalized probability embodiment 175b, risk score adder 142 computes general patient risk score 144 as a logarithmic summation of general independent vital sign risk scores 143a-143e in accordance with the following equation [15] for either the stable/non-deteriorating class C₀ or the unstable/deteriorating class C₁:

| | |
|---|---|
| GRS = Σlog (P(Xᵢ \| C₁)/P(Xᵢ)) | [15a] |
| GRS = log (P(C₁)/P(C_{O})) + Σlog (P(Xᵢ \| C₁)/P(Xᵢ)) | [15b] |

For equation [15b], log (P(C₁)/P(C₀)) again represents a term for biasing the GRS by the overall prevalence of unstable/deteriorating class C₁.

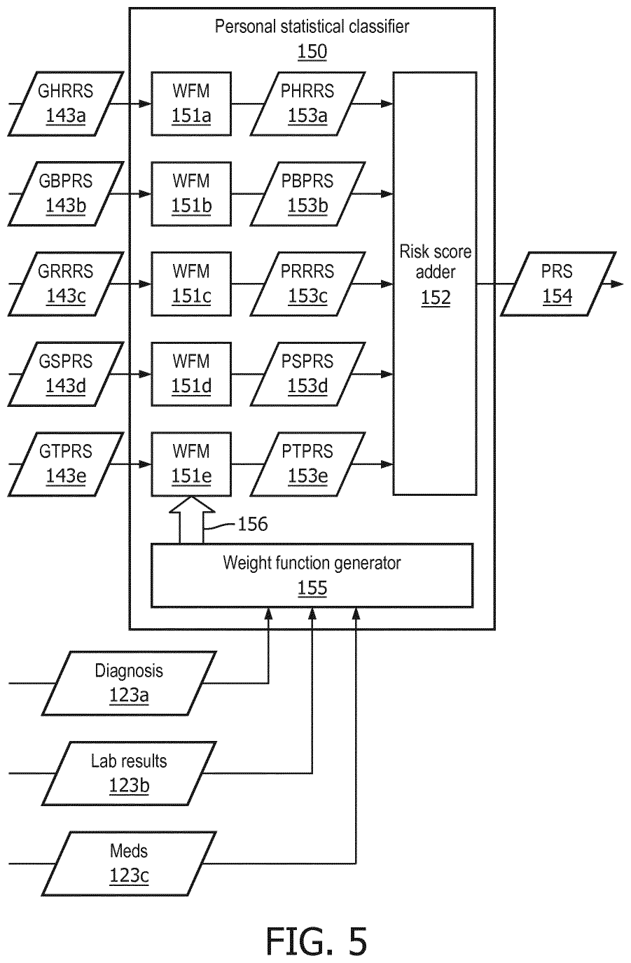

Referring to FIG. 5, one embodiment of personal statistical classifier (FIG. 1) is a personal statistical classifier 150 employing a parallel network of five (5) weighted function multipliers (WPM) 151a-151d, a risk score adder 152 and a weight function generator 155.

In practice, weighted function multiplier 151a is constructed and trained to input general heart rate risk score (GHRRS) 143a and plural weighted functions 156 to compute a personal heart rate risk score (PHRRS) 153a for each weighted function 156.

Weighted function multiplier 151b is constructed and trained to input general blood pressure risk score (GBPRS) 143b and the plural weighted functions 156 to thereby render a personal blood pressure risk score (PBPRS) 153b for each weighted function 156.

Weighted function multiplier 151c is constructed and trained to input general respiratory rate risk score (GRRRS) 143c and the plural weighted functions 156 to thereby render a personal respiratory rate risk score (PRRRS) 153c for each weighted function 156.

Weighted function multiplier 151d is constructed and trained to input general blood oxygen saturation risk score (GSPRS) 143d and the plural weighted functions 156 to thereby render a personal blood oxygen saturation risk score (PSPRS) 153d for each weighted function 156.

Weighted function multiplier 151e is constructed and trained to input general temperature risk score (GTPRS) 143e and the plural weighted functions 156 to thereby render a personal temperature risk score (PTPRS) 153e for each weighted function 156.

In practice, each weighted function multiplier 151 is any type of multiplier as known in the art prior to and subsequent to the present disclosure that is constructed in accordance with the principles of the present disclosure to compute personal independent vital sign risk scores 153a-153e as a product of a corresponding general independent vital sign risk scores 143a-143a and each of the plural weighted functions 156.

In practice, risk score adder 152 is any type of adder as known in the art prior to and subsequent to the present disclosure that is constructed accordance with the principles of the present disclosure to compute a personal patient risk score 154 as a logarithmic summation of personal heart rate risk scores 153a-153e.

In practice, weight matrix generator 155 is any type of arithmetic logic unit as known in the art prior to and subsequent to the present disclosure that is constructed in accordance with the principles of the present disclosure to generate a weighting function of diagnosis patient feature 123a informative of a cardiac clinical diagnosis, a lab results patient feature 123b informative of results of a cardiac laboratory test, and a medication patient feature 123c information of a prescribed cardiac medication.

In practice, weight matrix generator 155 encodes a patient feature and applies the encoded patient feature to a weighted coefficient that is priori determined through logistic regression with regularization during the training of personal statistical classifier 150 (FIG. 3A). In one embodiment, a logistic regression algorithm (e.g., a maximum likelihood estimation) is utilized to estimate the weighted coefficient from training data associated with the patient feature. For example, the weighted coefficient is modeled in a manner to predict a value very close to “0” for the stable/non-deteriorating class C₀ and a value very close to “1” for the unstable/deteriorating class C₁ to thereby seek a value of the weighted coefficient that minimizes an error in the probability predicted by the model to a probability delineated by the training data (e.g., minimize an error a probability of “0” if the training data and the patient feature corresponds to stable/non-deteriorating patient condition and minimize a probability of “1” if the training data and the patient feature corresponds to unstable/deteriorating patient condition).

Further in practice, weighted coefficient(s) for a particular patient feature 123 may be determined for all of the vital signs 141a-141e (FIG. 3A) or a set of weighted coefficients may be determined for a particular patient feature 123 on a vital sign basis.

In one embodiment, weight matrix generator 155 implements a binary encoding or one-hot encoding of categorical variable(s) or continuous variable(s) for each patient feature 123. For example, for diagnosis patient feature 123a, a binary encoding may be “0” for an absence of a categorical variable of a diagnosed cardiac disease and may be “1” for a presence of a categorical variable of a diagnosed cardiac disease. By further example, for lab results patient feature 123b, a one-hot encoding may be used for multiple continuous variables of results of a cardiac laboratory tests. By even further example, for medication patient feature 123c, a binary encoding may be “0” for a no-use categorical variable of a prescribed cardiac medication and may be “1” for a use categorical variable of a prescribed cardiac medication.

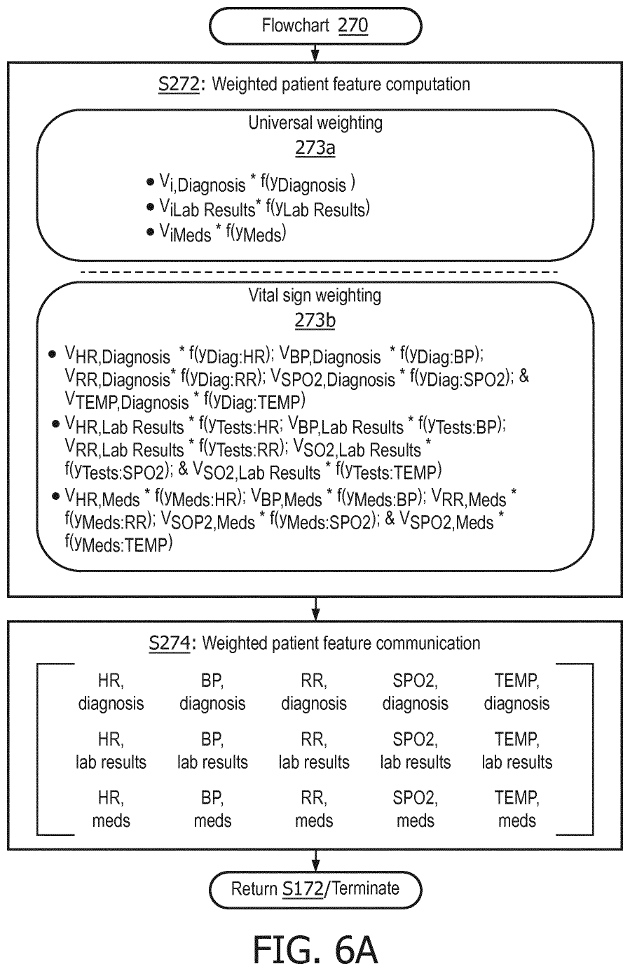

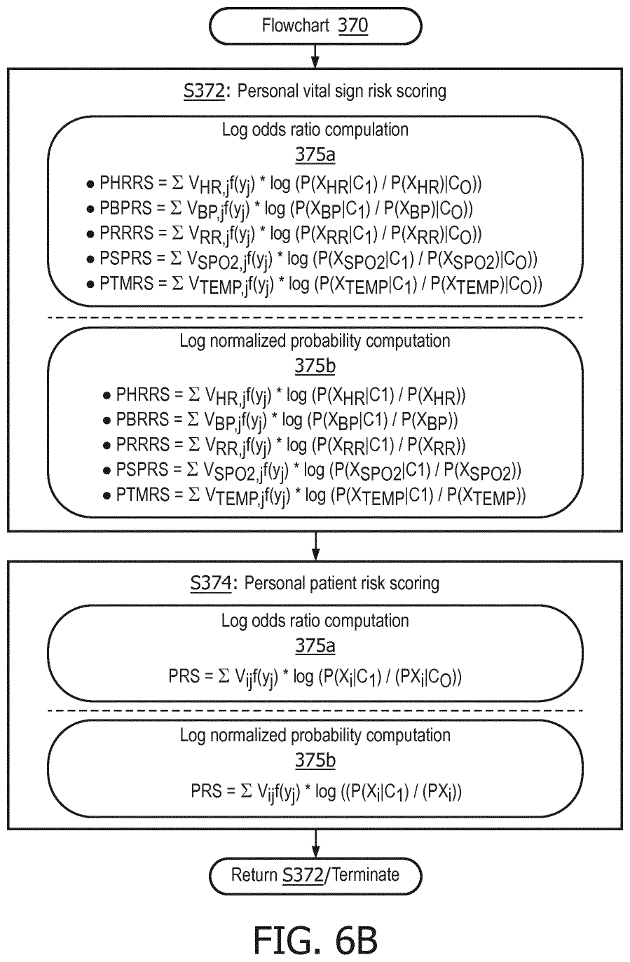

Referring to FIGS. 5, 6A and 6B, in operation, personal statistical classifier implements a flowchart 270 and a flowchart 370 representative of the personal patient risk scoring computation stage S74 of FIG. 2.

Referring to FIG. 6A, during a stage S272 of flowchart 270, weighted matrix generator 155 generate weighting functions Vᵢⱼ*f(yⱼ) from patient features 123a-123c, where f(yⱼ) is an encoded patient feature 123 and Vᵢⱼ is a priori trained weighted coefficient associated with the encoded patient feature 123.

In a universal weighting embodiment 273a, weighted matrix generator 155 generates a weighting coefficient V_{iDiagnosis}*f(y_{Diagnosis}) from diagnosis patient features 123a for all vital signs, a weighting coefficient V_{iLab Results}*f(y_{Lab Results}) from lab results patient features 123b for all vital signs, and a weighting coefficient V_{iMeds}*f(y_{Meds}) from meds patient features 123c for all vital signs.

In a vital sign embodiment 273b, for heart rate 112a (FIG. 3), weighted matrix generator 155 generates a weighting coefficient V_{HR,Diagnosis}*f(y_{Diagnosis:HR}) from diagnosis patient features 123a, a weighting coefficient V_{HR,Lab Results}*f(y_{LabResults:HR}) from lab results patient features 123b, and a weighting coefficient V_{HR,Meds}*f(y_{Meds:HR}) from meds patient features 123c.

For blood pressure 112b (FIG. 3), weighted matrix generator 155 generates a weighting coefficient V_{BP,Diagnosis}*f(y_{Diagnosis:BP}) from diagnosis patient features 123a, a weighting coefficient V_{BP,Lab Results}*f(y_{Lab Results:BP}) from lab results patient features 123b, and a weighting coefficient V_{BP,meds}*f(y_{meds:BP}) from meds patient features 123c.

For respiratory rate 112c (FIG. 3), weighted matrix generator 155 generates a weighting coefficient V_{RR,Diagnosis}*f(y_{Diagnosis:RR}) from diagnosis patient features 123a, a weighting coefficient V_{RR,Lab Results}*f(y_{Lab Results:RR}) from lab results patient features 123b, and a weighting coefficient V_{RR,Meds}*f(y_{Meds:RR}) from meds patient features 123c.

For blood oxygen saturation 112d (FIG. 3), weighted matrix generator 155 generates a weighting coefficient V_{SPO2,Diagnosis}*f(y_{Diagnosis:SPO2}) from diagnosis patient features 123a, a weighting coefficient V_{SPO2,Lab Results}*f(y_{Lab Results:SPO2}) from lab results patient features 123b, and a weighting coefficient V_{SPO2,Meds}*f(y_{Meds:SPO2}) from meds patient features 123c.

For temperature 112e (FIG. 3), weighted matrix generator 155 generates a weighting coefficient V_{TEMP,Diagnosis}*f(y_{Diagnosis:TEMP}) from diagnosis patient features 123a, a weighting coefficient V_{TEMP,Lab Results}*f(y_{Lab Results:TEMP}) from lab results patient features 123b, and a weighting coefficient V_{TEMP,Meds}*f(y_{Meds:TEMP}) from meds patient features 123c.

During a stage S274 of flowchart 270, weighted matrix generator 155 communicates the weighted functions Vᵢⱼ*f(yᵢ) to each weighted function multipliers 143a-143e. The communication follows a matrix of weighted functions Vᵢⱼ*f(yⱼ) arranged by columns of vital signs 112a-112e and rows of patient features 123a-123c as shown, or vice-versa.

Referring to FIG. 6B, during a stage S372 of flowchart 370, weighted function multipliers 151a-151e independently computes the plural personal independent vital sign risk scores 153a-153e respectively for the plural general independent vital sign risk scores 143a-143e.

For a log odds ratio embodiment 375a, weighted function multipliers 151a-151e independently computes personal independent vital sign risk scores 153a-153e respectively for general independent vital sign risk scores 143a-143e in accordance with the following equations [16]-[20]:

| | |
|---|---|
| PHHRS = V_{HR}, ⱼf(yⱼ)*log (P(X_{HR} \| C₁)/P(X_{HR} \| C₀)) | [16] |
| PBPRS = V_{BP}, ⱼf(yⱼ)*log (P(X_{BP} \| C₁)/P(X_{BP} \| C₀)) | [17] |
| PRRRS = V_{RR}, ⱼf(yⱼ)*log (P(X_{RR} \| C₁)/P(X_{RR} \| C₀)) | [18] |
| PSPRS = V_{SPO2}, ⱼf(yⱼ)*log (P(X_{SPO2} \| C₁)/P(X_{SPO2} \| C₀)) | [19] |
| PTPRS = V_{TEMP}, ⱼf(yⱼ)*log (P(X_{TEMP} \| C₁)/P(X_{TEMP} \| C₀)) | [20] |

For a normalized probability embodiment (FIG. 3C), weighted function multipliers 151a-151e independently computes the plural personal independent vital sign risk scores 153a-153e respectively for general independent vital sign risk scores 143a-143e in accordance with the following equations [21]-[25] for either the stable/non-deteriorating class C₀ or the unstable/deteriorating class C₁:

| | |
|---|---|
| PHHRS = V_{HR}, ⱼf(yⱼ)*log (P(X_{HR} \| C₁)/P(X_{HR})) | [21] |
| PBPRS = V_{BP}, ⱼf(yⱼ)*log (P(X_{BP} \| C₁)/P(X_{NP})) | [22] |
| PRRRS = V_{RR}, ⱼf(yⱼ)*log (P(X_{RR} \| C₁)/P(X_{RR})) | [23] |
| PSPRS = V_{SPO2}, ⱼf(yⱼ)*log (P(X_{SPO2} \| C₁)/P(X_{SPO2})) | [24] |
| PTPRS = V_{TEMP}, ⱼf(yⱼ)*log (P(X_{TEMP} \| C₁)/P(X_{TEMP})) | [25] |

During a stage S374 of flowchart 370, risk score adder 152 compute personal patient risk score 154 as a summation of the plural personal independent vital sign risk scores 153a-153e.

For log odds ratio embodiment 375a, risk score adder 152 computes personal patient risk score 154 as a summation of the plural personal independent vital sign risk scores 153a-153e in accordance with the following equation [26]:

PRS=ΣV_{i,j}f(yᵢ)*log(P(Xᵢ|C₁)/P(Xᵢ|C₀)) [26]

For a normalized probability embodiment 375b, risk score adder 152 computes personal patient risk score 154 as a logarithmic summation of personal independent vital sign risk scores 153a-153e in accordance with the following equation [27] for either the stable/non-deteriorating class C₀ or the unstable/deteriorating class C₁:

PRS=ΣV_{i,j}f(yⱼ)*log(P(Xᵢ|C₁)/P(Xᵢ) [27]

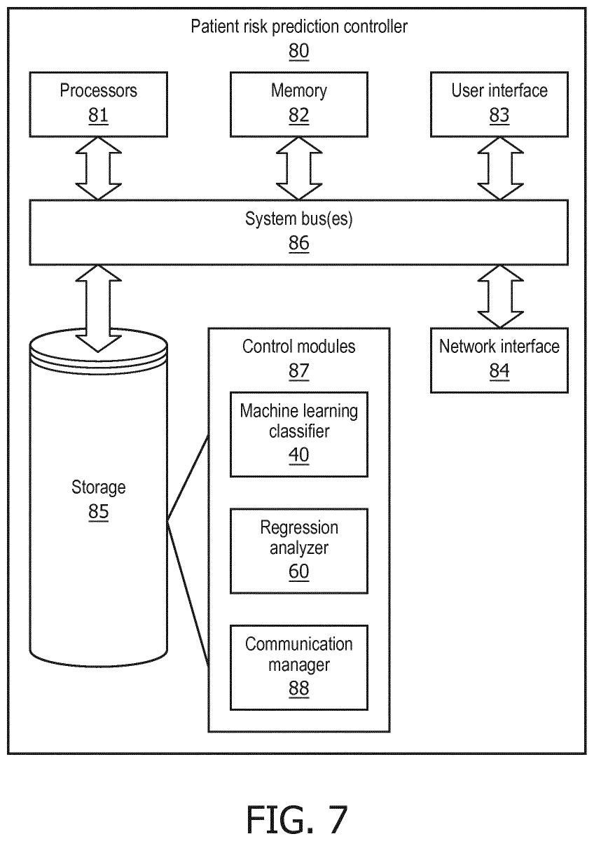

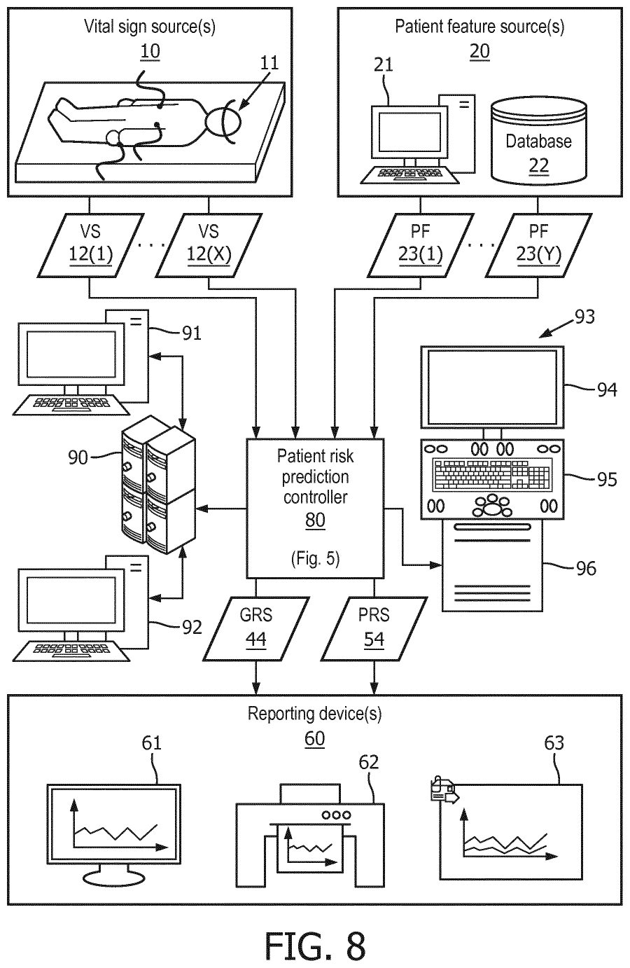

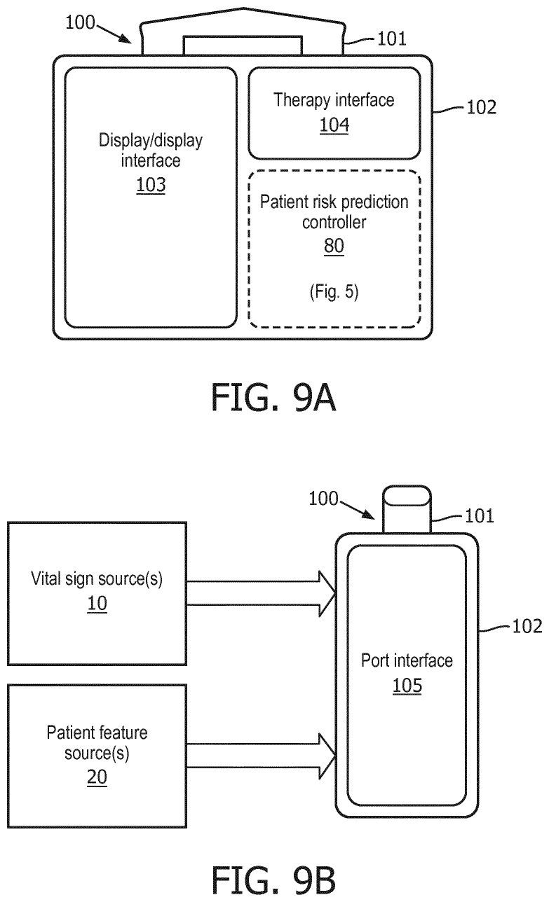

To further facilitate an understanding of the present disclosure, the following description of FIG. 7 teaches various embodiments of a patient risk prediction controller of the present disclosure, the following description of FIG. 8 teaches various embodiments of patient risk prediction system of the present disclosure, and the following description of FIGS. 9A and 9B teaches various embodiments of patient risk prediction device of the present disclosure. From the description of FIGS. 7-9B, those having ordinary skill in the art of the present disclosure will appreciate how to apply the present disclosure for making and using numerous and various additional embodiments of patient risk prediction controllers, patient risk prediction systems and patient risk prediction devices of the present disclosure.

In practice, a patient risk prediction controller of the present disclosure may be embodied as hardware/circuitry/software/firmware for implementation of a patient risk prediction method of the present disclosure as previously described herein. Further in practice, a patient risk prediction controller may be customized and installed in a server, workstation, etc. or programmed on a general purpose computer.

In one embodiment as shown in FIG. 7, a patient risk prediction controller 80 includes a processor 81, a memory 82, a user interface 83, a network interface 84, and a storage 85 interconnected via one or more system bus(es) 86. In practice, the actual organization of the components 81-85 of controller 80 may be more complex than illustrated.

The processor 81 may be any hardware device capable of executing instructions stored in memory or storage or otherwise processing data. As such, the processor 81 may include a microprocessor, field programmable gate array (FPGA), application-specific integrated circuit (ASIC), or other similar devices.

The memory 82 may include various memories such as, for example L1, L2, or L3 cache or system memory. As such, the memory 82 may include static random access memory (SRAM), dynamic RAM (DRAM), flash memory, read only memory (ROM), or other similar memory devices.

The user interface 83 may include one or more devices for enabling communication with a user such as an administrator. For example, the user interface 83 may include a display, a mouse, and a keyboard for receiving user commands. In some embodiments, the user interface 83 may include a command line interface or graphical user interface that may be presented to a remote terminal via the network interface 84.

The network interface 84 may include one or more devices for enabling communication with other hardware devices. For example, the network interface 84 may include a network interface card (NIC) configured to communicate according to the Ethernet protocol. Additionally, the network interface 84 may implement a TCP/IP stack for communication according to the TCP/IP protocols. Various alternative or additional hardware or configurations for the network interface will be apparent.

The storage 85 may include one or more machine-readable storage media such as read-only memory (ROM), random-access memory (RAM), magnetic disk storage media, optical storage media, flash-memory devices, or similar storage media. In various embodiments, the storage 85 may store instructions for execution by the processor 81 or data upon with the processor 81 may operate. For example, the storage 85 store a base operating system (not shown) for controlling various basic operations of the hardware.

More particular to the present disclosure, storage 85 may store control modules 87 in the form of general statistical classifier 40 (FIG. 1), personal statistical classifier 60 (FIG. 1) and a communication manager 88 (e.g., a display processor, a plotter manager and/or an email manager).

Referring to FIG. 8, in practice, patient risk prediction controller 80 may be installed/programmed within an application server 90 accessible by a plurality of clients (e.g., a client 91 and a client 92 as shown) and/or is installed/programmed within a workstation 93 employing a monitor 94, a keyboard 95 and a computer 96.

In operation, patient risk prediction controller 80 inputs medical imaging data 30, planar or volumetric, from medical imaging data sources 80 during a training phase and a phase. Medical imaging data sources 90 may include any number and types of medical imaging machines (e.g., a MRI machine 91, a CT machine 93, an X-ray machine 95 and an ultrasound machine 97 as shown) and may further includes database management/file servers (e.g., MRI database management server 92, CT server 94, X-ray database management server 96 and ultrasound database manager server 97 as shown). In practice, application server 90 or workstation 93, whichever is applicable, may be directly or networked connected to a medical imaging data source 90 to thereby input medical imaging data 30 for patient risk prediction controller 80. Alternatively, a medical imaging data source 90 and application server 90 or workstation 93, whichever is applicable, may be directly integrated whereby the patient risk prediction controller 80 has direct access to medical imaging data 30.

Referring to FIGS. 9A and 9B, a patient risk prediction device 100 (e.g., a defibrillator) of the present disclosure employs a handle 101 attached to a housing 102 providing user-access to a display/display interface 103, a therapy interface 104 and a port interface 105 Housing 12 further encloses patient risk prediction controller 80 in addition to other controllers (not shown) implementing additional functionality (e.g., synchronized shocking).

In practice, display/display interface 103 displays patient monitoring data as customized by a user via display interface 103 (e.g., keys) and patient risk score(s) as generated by patient risk prediction controller 80 as previously described in the present disclosure. Controller interface 15 (e.g., knobs and buttons) allows the user to apply various therapies (e.g., a shock) to a patient. Port interface 17 allows for the connection by the user to vital sign source(s) 10 for receiving vital signs and to patient feature sources (20) for receiving patient features.

Referring to FIGS. 1-9, those having ordinary skill in the art will appreciate the many benefits of the present disclosure including, but not limited to, systems, devices and methods adoptable by individual care providers and health care organizations for rendering a reliable patient risk prediction of a stable/non-deteriorating patient condition or an unstable/deteriorating patient condition.

Furthermore, it will be apparent that various information described as stored in the storage may be additionally or alternatively stored in the memory. In this respect, the memory may also be considered to constitute a “storage device” and the storage may be considered a “memory.” Various other arrangements will be apparent. Further, the memory and storage may both be considered to be “non-transitory machine-readable media.” As used herein, the term “non-transitory” will be understood to exclude transitory signals but to include all forms of storage, including both volatile and non-volatile memories.

While the device is shown as including one of each described component, the various components may be duplicated in various embodiments. For example, the processor may include multiple microprocessors that are configured to independently execute the methods described in the present disclosure or are configured to perform steps or subroutines of the methods described in the present disclosure such that the multiple processors cooperate to achieve the functionality described in the present disclosure. Further, where the device is implemented in a cloud computing system, the various hardware components may belong to separate physical systems. For example, the processor may include a first processor in a first server and a second processor in a second server.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the present disclosure is capable of other embodiments and its details are capable of modifications in various obvious respects. As is readily apparent to those skilled in the art, variations and modifications can be affected while remaining within the spirit and scope of the present disclosure. Accordingly, the foregoing disclosure, description, and figures are for illustrative purposes only and do not in any way limit the present disclosure, which is defined only by the claims.

1. A patient risk prediction controller, comprising:
a memory storing an artificial intelligence engine including:
a general statistical classifier trained on at least one vital sign to render at least one general independent vital sign risk score, and
a personal statistical classifier trained on at least one patient feature to render at least one personal independent vital sign risk score; and
at least one processor in communication with the memory, wherein the at least one processor is configured to perform at least one of the following steps:
(A) derive a singular personal independent vital sign risk score by:
(1) applying the general statistical classifier to a singular vital sign to render a singular general independent vital sign risk score, and
(2) applying the personal statistical classifier to the singular general independent vital sign risk score and a singular patient feature to derive the singular personal independent vital sign risk score from an integration of the singular patient feature into the singular general independent vital sign risk score; or
(B) derive a first plural personal independent vital sign risk scores by:
(1) applying the general statistical classifier to a singular vital sign to render a singular general independent vital sign risk score, and
(2) applying the personal statistical classifier to the singular general independent vital sign risk score and plural patient features to derive the first plural personal independent vital sign risk scores from an individual integration of each patient feature of the plural patient features into the singular general independent vital sign risk score; or
(C) derive a second plural personal independent vital sign risk scores by:
(1) applying the general statistical classifier to plural vital signs to render plural general independent vital sign risk scores, and
(2) applying the personal statistical classifier to the plural general independent vital sign risk scores and the singular patient feature to derive the second plural personal independent vital sign risk scores from an individual integration of the singular patient feature into each general independent vital sign risk score of the plural general independent vital sign risk scores; or
(D) derive a third plural personal independent vital sign risk scores by:
(1) applying the general statistical classifier to plural vital signs to render plural general independent vital sign risk scores, and
(2) apply the personal statistical classifier to the plural general independent vital sign risk scores and the plural patient features to derive the third plural personal independent vital sign risk scores from an individual integration of each patient feature of the plural patient features into each general independent vital sign risk score of the plural general independent vital sign risk scores.

2. The patient risk prediction controller of claim 1,
wherein the general statistical classifier is configured to quantify one or each of the at least one general independent vital sign risk score as a log odds ratio or a log normalized probability; and
wherein the personal statistical classifier is configured to quantify one or each of the at least one personal independent vital sign risk score is quantified as the log odds ratio or the log normalized probability.

3. The patient risk prediction controller of claim 1,
wherein the integration by the personal statistical classifier of the singular patient feature into the singular general independent vital sign risk score includes the personal statistical classifier configured to apply a weighted function of the singular patient feature to the singular general independent vital sign risk score;
wherein the individual integration by the personal statistical classifier of each patient feature of the plural patient features into the singular general independent vital sign risk score includes the personal statistical classifier configure to individual apply a weighted function of each patient feature to the singular general independent vital sign risk score;
wherein the individual integration by the personal statistical classifier of the singular patient feature into each general independent vital sign risk score of the plural general independent vital sign risk scores includes the personal statistical classifier configured to individually apply the weighted function of the singular patient feature to each plural general independent vital sign risk score; and
wherein the individual integration by the personal statistical classifier of the each patient feature of the plural patient features into each general independent vital sign risk score of the plural general independent vital sign risk scores includes the personal statistical classifier configured to individually apply the weighted function of each patient feature to each plural general independent vital sign risk score.

4. The patient risk prediction controller of claim 1,
wherein the general statistical classifier is configured to compute a general patient risk score from one of the singular general independent vital sign risk or the plural general independent vital sign risk scores; and
wherein the at least one processor is further configured to control a computation of the general patient risk score by the general statistical classifier.

5. The patient risk prediction controller of claim 4, wherein the general statistical classifier is configured to compute the general patient risk score as one of an equivalent of the singular general independent vital sign risk or a summation of the plural general independent vital sign risk scores.

6. The patient risk prediction controller of claim 1,
wherein the personal statistical classifier is configured to compute a personal patient risk score from one of: the singular personal independent vital sign risk, the first plural personal independent vital sign risk scores, the second plural personal independent vital sign risk scores, or the third plural personal independent vital sign risk scores; and
wherein the at least one processor is further configured to control a computation of the personal patient risk score by the personal statistical classifier.

7. The patient risk prediction controller of claim 6, wherein the personal statistical classifier is configured to compute the personal patient risk score as one of an equivalent of the singular personal independent vital sign risk or a summation of one of the: first plural personal independent vital sign risk scores, second plural personal independent vital sign risk scores, or third plural personal independent vital sign risk scores.

8. The patient risk prediction controller of claim 1,
wherein at least one of:
the general statistical classifier is configured to derive a general patient risk score from one of the singular general independent vital sign risk score or the plural general independent vital sign risk scores, or
the personal statistical classifier is configured to derive a personal patient risk score from one of the singular personal vital sign risk score, the first plural personal independent vital sign risk scores, the second plural personal independent vital sign risk scores, or the third plural personal vital sign risk scores;
wherein the artificial intelligence engine further includes a communication manager; and
wherein the at least one processor is further configured to:
control a communication by the communication manager of at least one of: the general patient risk score or the personal patient risk score to at least one reporting device.

9. A non-transitory machine-readable storage medium encoded with instructions for execution by at least one processor of an artificial intelligence engine including a general statistical classifier and a personal statistical classifier,
the general statistical classifier trained on at least one vital sign to render at least one general independent vital sign risk score,
the personal statistical classifier trained on at least one patient feature to render at least one personal independent vital sign risk score,
the non-transitory machine-readable storage medium comprising instructions to perform at least one of:
(A) derive a singular personal independent vital sign risk score by:
(1) applying the general statistical classifier to a singular vital sign to render a singular general independent vital sign risk score, and
(2) applying the personal statistical classifier to the singular general independent vital sign risk score and a singular patient feature to derive the singular personal independent vital sign risk score from an integration of the singular patient feature into the singular general independent vital sign risk score; or
(B) derive a first plural personal independent vital sign risk scores by:
(1) applying the general statistical classifier to a singular vital sign to render a singular general independent vital sign risk score, and
(2) applying the personal statistical classifier to the singular general independent vital sign risk score and plural patient features to derive the first plural personal independent vital sign risk scores from an individual integration of each patient feature of the plural patient features into the singular general independent vital sign risk score; or
(C) derive a second plural personal independent vital sign risk scores by:
(1) applying the general statistical classifier to plural vital signs to render plural general independent vital sign risk scores, and
(2) applying the personal statistical classifier to the plural general independent vital sign risk scores and the singular patient feature to derive the second plural personal independent vital sign risk scores from an individual integration of the singular patient feature into each general independent vital sign risk score of the plural general independent vital sign risk scores; or
(D) derive a third plural personal independent vital sign risk scores by:
(1) applying the general statistical classifier to plural vital signs to render plural general independent vital sign risk scores, and
(2) apply the personal statistical classifier to the plural general independent vital sign risk scores and the plural patient features to derive the third plural personal independent vital sign risk scores from an individual integration of each patient feature of the plural patient features into each general independent vital sign risk score of the plural general independent vital sign risk scores.

10. The non-transitory machine-readable storage medium of claim 9,
wherein one or each of the at least one general independent vital sign risk score is quantified as a log odds ratio or a log normalized probability; and
wherein one or each of the at least one personal independent vital sign risk score is quantified as the log odds ratio or the log normalized probability.

11. The non-transitory machine-readable storage medium of claim 9,
wherein the instruction to integrate the singular patient feature into the singular general independent vital sign risk score includes instructions to apply a weighted function of the singular patient feature to the singular general independent vital sign risk score;
wherein the instruction to individually integrate each patient feature of the plural patient features into the singular general independent vital sign risk score includes instructions to individually apply a weighted function of each patient feature to the singular general independent vital sign risk score;
wherein the instruction to individually integrate the singular patient feature into each general independent vital sign risk score of the plural general independent vital sign risk scores includes instructions to individually apply the weighted function of the singular patient feature to each plural general independent vital sign risk score; and
wherein the instruction to individually integrate each patient feature of the plural patient features into each general independent vital sign risk score of the plural general independent vital sign risk scores includes instructions to individually apply the weighted function of each patient feature to each plural general independent vital sign risk score.

12. The non-transitory machine-readable storage medium of claim 9, wherein the non-transitory machine-readable storage medium further includes instructions to:
compute, via the general statistical classifier, a general patient risk score from one of the singular general independent vital sign risk or the plural general independent vital sign risk scores.

13. The non-transitory machine-readable storage medium of claim 12, wherein the general patient risk score is one of an equivalent of the singular general independent vital sign risk or an aggregation of the plural general independent vital sign risk scores.

14. The non-transitory machine-readable storage medium of claim 9, wherein the non-transitory machine-readable storage medium further includes instructions to:
compute, via the personal statistical classifier, a personal patient risk score from one of: the singular personal independent vital sign risk, the first plural personal independent vital sign risk scores, the second plural personal independent vital sign risk scores, or the third plural personal independent vital sign risk scores.

15. The non-transitory machine-readable storage medium of claim 14, wherein the personal patient risk score is one of an equivalent of the singular personal independent vital sign risk or an aggregation of one of: the first plural personal independent vital sign risk scores, the second plural personal independent vital sign risk scores, or the third plural personal independent vital sign risk scores.

16. A patient risk prediction method executable by an artificial intelligence engine including a general statistical classifier and a personal statistical classifier,
the general statistical classifier trained on at least one vital sign to render at least one general independent vital sign risk score,
the personal statistical classifier trained on at least one patient feature to render at least one personal independent vital sign risk score,
the patient risk prediction method comprising at least one of:
(A) deriving a singular personal independent vital sign risk score by:
(1) applying the general statistical classifier to a singular vital sign to render a singular general independent vital sign risk score, and
(2) applying the personal statistical classifier to the singular general independent vital sign risk score and a singular patient feature to derive the singular personal independent vital sign risk score from an integration of the singular patient feature into the singular general independent vital sign risk score; or
(B) deriving a first plural personal independent vital sign risk scores by:
(1) applying the general statistical classifier to a singular vital sign to render a singular general independent vital sign risk score, and
(2) applying the personal statistical classifier to the singular general independent vital sign risk score and plural patient features to derive the first plural personal independent vital sign risk scores from an individual integration of each patient feature of the plural patient features into the singular general independent vital sign risk score; or
(C) deriving a second plural personal independent vital sign risk scores by:
(1) applying the general statistical classifier to plural vital signs to render plural general independent vital sign risk scores, and
(2) applying the personal statistical classifier to the plural general independent vital sign risk scores and the singular patient feature to derive the second plural personal independent vital sign risk scores from an individual integration of the singular patient feature into each general independent vital sign risk score of the plural general independent vital sign risk scores; or
(D) deriving a third plural personal independent vital sign risk scores by:
(1) applying the general statistical classifier to plural vital signs to render plural general independent vital sign risk scores, and
(2) apply the personal statistical classifier to the plural general independent vital sign risk scores and the plural patient features to derive the third plural personal independent vital sign risk scores from an individual integration of each patient feature of the plural patient features into each general independent vital sign risk score of the plural general independent vital sign risk scores.

17. The patient risk prediction method of claim 16,
wherein one or each of the at least one general independent vital sign risk score is quantified as a log odds ratio or a log normalized probability; and
wherein one or each of the at least one personal independent vital sign risk score is quantified as the log odds ratio or the log normalized probability.

18. The patient risk prediction method of claim 16,
wherein the integrating, via the personal statistical classifier, of the singular patient feature into the singular general independent vital sign risk score includes applying, via the personal statistical classifier, a weighted function of the singular patient feature to the singular general independent vital sign risk score;
wherein the individual integrating, via the personal statistical classifier, of each patient feature of the plural patient features into the singular general independent vital sign risk score includes individually applying, via the personal statistical classifier, a weighted function of each patient feature to the singular general independent vital sign risk score;
wherein the individual integrating, via the personal statistical classifier, of the singular patient feature into each general independent vital sign risk score of the plural general independent vital sign risk scores includes individually applying the weighted function of the singular patient feature to each plural general independent vital sign risk score; and
wherein the individual integrating, via the personal statistical classifier, of each patient feature of the plural patient features into each general independent vital sign risk score of the plural general independent vital sign risk scores includes individually applying the weighted function of each patient feature to each plural general independent vital sign risk score.

19. The patient risk prediction method of claim 16, further comprising at least one of:
computing, via the general statistical classifier, a general patient risk score as an equivalent of the singular general independent vital sign risk; or
computing, via the general statistical classifier, the general patient risk score as an aggregation of the plural general independent vital sign risk scores.

20. The patient risk prediction method of claim 16, further comprising at least one of:
computing, via the personal statistical classifier, a personal patient risk score as an equivalent of the singular personal independent vital sign risk; or
computing, via the personal statistical classifier, the personal patient risk score as an aggregation of one of: the first plural personal independent vital sign risk scores, the second plural personal independent vital sign risk scores, or the third plural personal independent vital sign risk scores.
